SEQUENCE LISTING (6 sequences)

SEQ ID NO: 1 - RNA, 12 nt, Artificial sequence
  Synthetic oligonucleotide
caggucugugau

SEQ ID NO: 2 - DNA, 20 bp, Artificial sequence
  Synthetic oligonucleotide
tccatgacgttcctgatgct

SEQ ID NO: 3 - DNA, 19 bp, Artificial sequence
  Synthetic oligonucleotide
gggggacgatcgtcggggg

SEQ ID NO: 4 - RNA, 20 nt, Artificial sequence
  Synthetic oligonucleotide
gcccgacagaagagagacac

SEQ ID NO: 5 - RNA, 20 nt, Artificial sequence
  Synthetic oligonucleotide
acccaucuauuauauaacuc

SEQ ID NO: 6 - RNA, 20 nt, Artificial sequence
  Synthetic oligonucleotide
gcccgucuguugugugacuc

(19) United States
(12) Patent Application Publication
(10) Pub. No.: US 2009/0214578 A1
(43) Pub. Date: Aug. 27, 2009

(54) Immunostimulatory Single-Stranded Ribonucleic Acid with Phosphodiester Backbone

(75) Inventor: Stefan Bauer, Marburg-Michellbach (DE)

Correspondence Address: WOLF GREENFIELD & SACKS, P.C., 600 ATLANTIC AVENUE, BOSTON, MA, 02210-2206, US

(73) Assignee: Coley Pharmaceutical GmbH, Duesseldorf (DE)

(21) Appl. No.: 11/992,080

(22) PCT Filed: Sep. 15, 2006
(86) PCT No.: PCT/IB2006/004169

§ 371 Date: May 12, 2009

Related U.S. Application Data
(60) Provisional application No. 60/718,087, filed on Sep. 16, 2005.

(51) Int. Cl. A61K 39/00; A61K 31/7052; A61P 37/04; A61P 35/00; A61P 31/00; A61P 11/06

(52) U.S. Cl. 424/184.1; 514/44 R

(57) ABSTRACT

Immunostimulatory single-stranded oligoribonucleotides (ssORN) with phosphodiester backbones induce TLR7-independent and MyD88-dependent immune activation. These immunostimulatory ssORN are useful to induce a ThI-like immune response in a subject, to induce an antigen-specific immune response in a subject, and to treat a subject having a cancer, an infectious disease, an allergic condition, or asthma.

### BACKGROUND OF THE INVENTION

Ribonucleic acid (RNA) has recently been the focus of intense interest because of its newly-recognized potential as a therapeutic. It has recently been reported, for example, that certain sequence-specific double-stranded RNA, generally about 21-23 nucleotides long, can be used to silence gene expression in a selective manner, in a process called RNA interference (RNAi) or post-transcriptional gene silencing. Double-stranded RNA used for this type of RNA interference includes, in particular, so-called short interfering RNA (siRNA). Hannon G J (2002) Nature 418:244-51. In contrast, it has also recently been reported that sequence-nonspecific double-stranded RNA can induce immunostimulatory effects, acting through Toll-like receptor 3 (TLR3). Alexopoulou L et al. (2001) Nature 413:732-8. Further, it has also been recently reported that certain single-stranded RNAs, generally including guanosine (G) and uridine (U), and particularly including certain sequence motifs, are also immunostimulatory. Lipford et al. US 2003/0232074 A1. Immunostimulatory single-stranded RNA have been reported to act through Toll-like receptor 7 (TLR7) and Toll-like receptor 8 (TLR8).

In addition to immune stimulation arising through interaction of RNA with TLR3, TLR7, and TLR8, certain cytosine-guanine dinucleotide (CpG)-containing nucleic acid molecules, in particular CpG-containing deoxyribonucleic acid (DNA), have been reported to exert their immunostimulatory effect through interaction with Toll-like receptor 9 (TLR9). Hemmi H et al. (2000) Nature 408:740-5. TLR7, TLR8, and TLR9 all signal in an MyD88-dependent manner.

Deoxyribonucleic acid molecules and, even more so, ribonucleic acid molecules with naturally occurring phosphodiester internucleotide linkages in their sugar phosphate backbone, are susceptible to nuclease-mediated degradation. Since for clinical use immunostimulatory nucleic acids are frequently prepared as synthetic oligonucleotides, these immunostimulatory synthetic oligonucleotides frequently include one or more stabilized internucleotide linkages in their sugar phosphate backbone. A commonly used stabilized internucleotide linkage is phosphorothioate.

### SUMMARY OF THE INVENTION

It has now been discovered according to the invention that, surprisingly, single-stranded RNA with phosphodiester backbone, but not phosphorothioate backbone, stimulates immune activation at least in part through an MyD88-dependent Toll-like receptor other than TLR7 or TLR8. The MyD88-dependent Toll-like receptor responsible for this immune response is putatively assigned to be TLR9.

CpG-mediated immune activation, acting through TLR9, involves activation of innate immunity and leads to skewing of an immune response toward a Th1- or Th1-like immune response. CpG oligonucleotides thus have been reported to be useful as adjuvants and as active agents for use in the treatment of diseases where stimulation of a Th1 immune response is desired, e.g., cancer, infection, allergy, and asthma.

Accordingly, it has now been discovered according to the invention that, surprisingly, single-stranded RNA with phosphodiester backbone, without either CpG or sequence-specific features of previously described immunostimulatory single-stranded RNA, can be used in any application calling for TLR9-mediated immune system activation. Such applications include, without limitation, treatment of a subject having cancer, infection, allergy, or asthma.

In one aspect the invention is a method of inducing a Th1-like immune response in a subject. The method according to this aspect of the invention includes the step of administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one aspect the invention is a method of inducing an antigen-specific response in a subject. The method according to this aspect of the invention includes the steps of administering to the subject an antigen; and administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one aspect the invention is a method of treating a subject having a cancer. The method according to this aspect of the invention includes the step of administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one aspect the invention is a method of treating a subject having an infectious disease. The method according to this aspect of the invention includes the step of administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG Motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one aspect the invention is a method of treating a subject having an allergic condition. The method according to this aspect of the invention includes the step of administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one aspect the invention is a method of treating a subject having asthma. The method according to this aspect of the invention includes the step of administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that

(1) is free of guanosine (G), or

(2) comprises at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

In one embodiment the immunostimulatory ssORN is 5-40 nucleotides long.

In one embodiment the immunostimulatory ssORN is 5-20 nucleotides long.

In one embodiment the immunostimulatory ssORN is 5-12 nucleotides long.

In one embodiment the immunostimulatory ssORN is a synthetic ssORN.

In one embodiment the immunostimulatory ssORN is not a poly-nucleotide selected from the group consisting of poly-U, poly-G, poly-A, or poly-C.

In one embodiment the administering to the subject the effective amount of the immunostimulatory ssORN is systemically administering to the subject an effective amount of the immunostimulatory ssORN.

### BRIEF DESCRIPTION OF THE DRAWINGS

FIG. 1 is a schematic drawing depicting certain Toll-like receptors, their ligands, and features of their intracellular signaling pathways, as previously understood. MyD88 is depicted as an adapter protein for nucleic acid-responsive Toll-like receptors TLR7, TLR8, and TLR9, as well as for non-nucleic acid-responsive Toll-like receptors TLR2, TLR4, and TLR5.

FIG. 2 is a graph depicting TLR7-independent recognition of single-stranded phosphodiester RNA. PD, phosphodiester backbone; PTO, phosphorothioate backbone; WT, wild-type. RNA63 is an oligoribonucleotide having a nucleotide sequence provided as 5′-CAGGUCUGUGAU-3′ (SEQ ID NO:1). CpG-ODN 1668 is an oligodeoxynucleotide having a nucleotide sequence provided as 5′-TCCATGACGTTCCTGATGCT-3′ (SEQ ID NO:2).

FIG. 3 is a group of three graphs depicting secretion of various indicated cytokines by FLT3-L-induced dendritic cells (DC) in response to various stimuli. LPS, lipopolysaccharide. CpG-ODN 2216 is an oligodeoxynucleotide having a nucleotide sequence provided as 5′-GGGGGACGATCGTCGGGGG-3′ (SEQ ID NO:3).

FIG. 4 is a series of four graphs depicting FACS analyses for CD69 expression on FLT3-L-induced dendritic cells in response to the indicated ssORN.

FIG. 5 is a pair of graphs depicting IL-12p40 secretion by M-CSF-derived macrophages and by GM-CSF-derived dendritic cells, in response to indicated stimuli.

FIG. 6A is a graph depicting IL-12p40 secretion of FLT3-L-induced dendritic cells from wild-type (WT), TLR7-knockout (TLR7−/−), and MyD88-knockout (MyD88−/−) mice, in response to indicated stimuli. pI:C, poly inosine:cytidine.

FIG. 6B is a series of three graphs depicting FACS analyses for CD69 expression by FLT3-L-induced dendritic cells from wild-type (WT), TLR7-knockout (TLR7−/−), and MyD88-knockout (MyD88−/−) mice, in response to indicated stimuli.

### DETAILED DESCRIPTION OF THE INVENTION

The immune response is conceptually divided into innate immunity and adaptive immunity. Innate immunity is believed to involve recognition of pathogen-associated molecular patterns (PAMPs) shared in common by certain classes of molecules expressed by infectious microorganisms or foreign macromolecules. PAMPs are believed to be recognized by pattern recognition receptors (PRRs) on certain immune cells.

Toll-like receptors (TLRs) are a family of highly conserved polypeptides that play a critical role in innate immunity in mammals. Currently ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adapter protein MyD88 has been reported to associate with many of the TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

While a number of specific TLR ligands have been reported, ligands for some TLRs remain to be identified. Ligands for TLR2 include peptidoglycan and lipopeptides. Yoshimura A et al. (1999) J Immunol 163:1-5; Yoshimura A et al. (1999) J Immunol 163:1-5; Aliprantis A O et al. (1999) Science 285:736-9. Viral-derived double-stranded RNA (dsRNA) and poly I:C, a synthetic analog of dsRNA, have been reported to be ligands of TLR3. Alexopoulou L et al. (2001) Nature 413:732-8. Lipopolysaccharide (LPS) is a ligand for TLR4. Poltorak A et al. (1998) Science 282:2085-8; Hoshino K et al. (1999) J Immunol 162:3749-52. Bacterial flagellin is a ligand for TLR5. Hayashi F et al. (2001) Nature 410:1099-1103. Peptidoglycan has been reported to be a ligand not only for TLR2 but also for TLR6. Ozinsky A et al. (2000) Proc Natl Acad Sci USA 97:13766-71; Takeuchi O et al. (2001) Int Immunol 13:933-40. Single-stranded RNA containing guanosine and uridine has been reported to be a ligand for TLR7 and TLR8. U.S. Pat. Appl. Pub. 2003/0232074 A1. Certain low molecular weight synthetic compounds, the imidazoquinolones imiquimod (R-837) and resiquimod (R-848), have also been reported to be ligands of TLR7 and TLR8. Jurk M et al. (2002) Nat Immunol 3:499; Hemmi H et al. (2002) Nat Immunol 3:196-200. Bacterial DNA (CpG DNA) has been reported to be a TLR9 ligand. Hemmi H et al. (2000) Nature 408:740-5; Bauer S et al. (2001) Proc Natl Acad Sci USA 98, 9237-42. The natural ligands for TLR1 and TLR10 are not known.

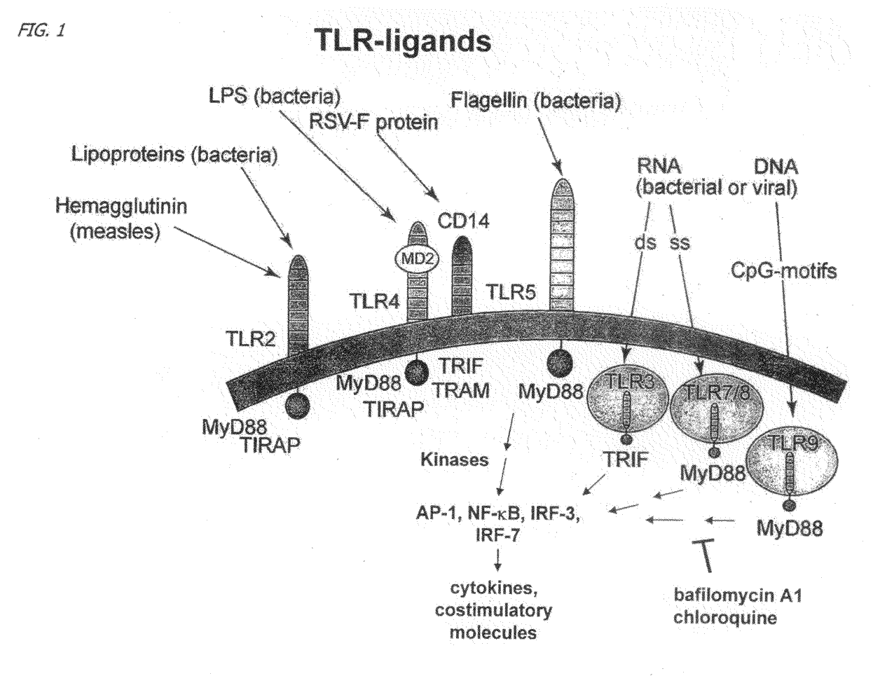

TLR7, TLR8, and TLR9 all signal in response to appropriate nucleic acid ligand with the participation of the adapter protein MyD88. Murine TLR8, unlike human TLR8, is thought to be nonfunctional. Thus in the mouse TLR7 and TLR9 are functional and signal the in response to appropriate nucleic acid ligand with the participation of MyD88. Mice lacking functional TLR7 thus have only TLR9 as a functional TLR known to be capable of signaling in response to appropriate nucleic acid ligand through an MyD88-dependent pathway (see FIG. 1).

It has now been surprisingly discovered that certain single-stranded oligoribonucleotides with phosphodiester, but not phosphorothioate, backbone are capable of stimulating immune cells in an MyD88-dependent manner in mice lacking functional TLR7 or TLR8.

Immunostimulatory single-stranded oligoribonucleotide (ssORN) useful according to the present invention are 5-100 nucleotides long, have a phosphodiester backbone and a nucleotide sequence that

(1) is free of guanosine (G), or

(2) includes at least one G, with proviso that when the nucleotide sequence comprises at least one G, the nucleotide sequence is

(a) free of uridine (U), and

(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,

with proviso that the immunostimulatory ssORN is not present as part of a double-stranded ribonucleic acid (RNA) molecule.

Immunostimulatory single-stranded oligoribonucleotide (ssORN) useful according to the present invention in one embodiment can be derived and isolated from natural sources of RNA. Alternatively, and more typically, immunostimulatory single-stranded oligoribonucleotide (ssORN) useful according to the present invention in one embodiment can be obtained by synthetic methods well known in the art.

The immunostimulatory ssORN of the invention can be of natural or non-natural origin. RNA as it occurs in nature is a type of nucleic acid that generally refers to a linear polymer of certain ribonucleoside units, each ribonucleoside unit made up of a purine or pyrimidine base and a ribose sugar, linked by internucleoside phosphodiester bonds. In this regard “linear” is meant to describe the primary structure of RNA. RNA in general can be single-stranded or double-stranded, including partially double-stranded.

As used herein, “nucleoside” refers to a single sugar moiety (e.g., ribose or deoxyribose) linked to an exchangeable organic base, which is either a substituted pyrimidine (e.g., cytosine, thymine, or uracil) or a substituted purine (e.g., adenine or guanine). Corresponding nucleotides are cytidine, thymidine, uridine, adenosine, and guanosine, which are conventionally denoted as C, T, U, A, and G, respectively. As described herein, the nucleoside may be a naturally occurring nucleoside, a modified nucleoside, or a synthetic (artificial) nucleoside.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, described above. As used herein, the terms refer to oligoribonucleotides (ORN) as well as oligodeoxyribonucleotides (ODN). The terms shall also include polynucleosides (i.e., a polynucleotide minus the phosphate) and any other organic base-containing polymer. Nucleic acid molecules can be obtained from existing nucleic acid sources (e.g., genomic or cDNA), but are preferably synthetic (e.g., produced by nucleic acid synthesis).

The terms nucleic acid and oligonucleotide also encompass nucleic acids or oligonucleotides with substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide nucleic acids (which have amino acid backbone with nucleic acid bases). In some embodiments, the nucleic acids are homogeneous in backbone composition. Nucleic acids also include substituted purines and pyrimidines such as C-5 propyne modified bases. Wagner R W et al. (1996) Nat Biotechnol 14:8404. Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

A natural nucleoside base can be replaced by a modified nucleoside base, wherein the modified nucleoside base is for example selected from hypoxanthine; dihydrouracil; pseudouracil; 2-thiouracil; 4-thiouracil; 5-aminouracil; 5-(C₁-C₆)-alkyluracil; 5-(C₂-C₆)-alkenyluracil; 5-(C₂-C₆)-alkynyluracil; 5-(hydroxymethyl)uracil; 5-chlorouracil; 5-fluorouracil; 5-bromouracil; 5-hydroxycytosine; 5-(C₁-C₆)-alkylcytosine; 5-(C₂-C₆)-alkenylcytosine; 5-(C₂-C₆)-alkynylcytosine; 5-chlorocytosine; 5-fluorocytosine; 5-bromocytosine; N²-dimethylguanine; 2,4-diamino-purine; 8-azapurine (including, in particular, 8-azaguanine); a substituted 7-deazapurine (including, in particular, 7-deazaguanine), including 7-deaza-7-substituted and/or 7-deaza-8-substituted purine; or other modifications of a natural nucleoside bases. This list is meant to be exemplary and is not to be interpreted to be limiting.

In particular, when there is at least one guanosine present in the immunostimulatory ssORN, at least one guanine base of the immunostimulatory ssORN can be a substituted or modified guanine such as 7-deazaguanine; 8-azaguanine; 7-deaza-7-substituted guanine (such as 7-deaza-7-(C2-C6)alkynylguanine); 7-deaza-8-substituted guanine; hypoxanthine; 2,6-diaminopurine; 2-aminopurine; purine; 8-substituted guanine such as 8-hydroxyguanine; and 6-thioguanine. This list is meant to be exemplary and is not to be interpreted to be limiting.

Also in particular, when there is at least one uridine present in the immunostimulatory ssORN, the at least one uracil base of the immunostimulatory ssORN can be a substituted or modified uracil such as pseudouracil and 5-methyluracil.

For use in the instant invention, the nucleic acids of the invention can be synthesized de novo using any of a number of procedures well known in the art. For example, the β-cyanoethyl phosphoramidite method (Beaucage S L et al. (1981) Tetrahedron Lett 22:1859); nucleoside H-phosphonate method (Garegg et al. (1986) Tetrahedron Lett 27:4051-4; Froehler et al. (1986) Nucl Acid Res 14:5399-407; Garegg et al. (1986) Tetrahedron Lett 27:4055-8; Gaffney et al. (1988) Tetrahedron Lett 29:2619-22). These chemistries can be performed by a variety of automated nucleic acid synthesizers available in the market. These nucleic acids are referred to as synthetic nucleic acids. Nucleic acids can be prepared from existing nucleic acid sequences (e.g., ribosomal, messenger, or transfer RNA) using known techniques, such as those employing restriction enzymes, exonucleases or endonucleases. Nucleic acids prepared in this manner are referred to as isolated nucleic acid. An isolated nucleic acid generally refers to a nucleic acid which is separated from components with which it is normally associated in nature. As an example, an isolated nucleic acid may be one which is separated from a cell, from a nucleus, from mitochondria or from chromatin. The term “nucleic acid” encompasses both synthetic and isolated nucleic acid.

The ssORN useful according to the invention are immunostimulatory. As used herein, an immunostimulatory ssORN refers to a ssORN that is capable of inducing an immune response, e.g., stimulating a cell of the immune system to become activated to proliferate, differentiate, migrate, increase cytolytic activity, increase expression of secreted products associated with immune cell activation, increase expression of cell surface markers or co-stimulatory molecules associated with immune cell activation, or any combination thereof. Secreted products associated with immune cell activation are well known in the art and can include, without limitation, cytokines, chemokines, and antibodies.

The immunostimulatory ssORN of the invention can be used to induce a Th1-like immune response, both in vitro and in vivo. As used herein, a Th1-like immune response refers to activation of immune cells to express Th1-like secreted products; including certain cytokines, chemokines, and subclasses of immunoglobulin; and activation of certain immune cells. Th1-like secreted products include, without limitation, the cytokines IFN-γ, IL-2, IL-12, IL-18, TNF-α, and the chemokine IP-10 (CXCL10). In the mouse, Th1 immune activation stimulates secretion of IgG2a. In the human, Th1 immune activation stimulates secretion of IgG1. Accordingly, a Th1-like immune response in a mouse can include increased secretion of IgG2a, and a Th1-like immune response in a human can include increased secretion of IgG1. Th1 and Th1-like immune activation also may include activation of NK cells and dendritic cells, i.e., cells involved in cellular immunity. Th1 and Th1-like immune activation are believed to counter-regulate Th2 immune activation.

The immunostimulatory ssORN of the invention can be used to induce an antigen-specific immune response, both in vitro and in vivo. As used herein, an antigen-specific immune response is a an adaptive immune response arising from contact between cells of the immune system and an antigen.

The term “antigen” refers to a molecule capable of provoking an immune response. The term antigen broadly includes any type of molecule that is recognized by a host system as being foreign. Antigens include but are not limited to microbial antigens, cancer antigens, and allergens. Antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many antigens are protein or polypeptide in nature, as proteins and polypeptides are generally more antigenic than carbohydrates or fats.

The antigen can be an antigen that is encoded by a nucleic acid vector or it can be an antigen per se. In the former case the nucleic acid vector is administered to the subject and the antigen is expressed in vivo. In the latter case the antigen may be administered directly to the subject. An antigen not encoded in a nucleic acid vector as used herein refers to any type of antigen that is not a nucleic acid. For instance, in some aspects of the invention the antigen not encoded in a nucleic acid vector is a peptide or a polypeptide. Minor modifications of the primary amino acid sequences of peptide or polypeptide antigens may also result in a polypeptide which has substantially equivalent antigenic activity as compared to the unmodified counterpart polypeptide. Such modifications may be deliberate, as by site-directed mutagenesis, or may be spontaneous. All of the polypeptides produced by these modifications are included herein as long as antigenicity still exists. The peptide or polypeptide may be, for example, virally derived. The antigens useful in the invention may be any length, ranging from small peptide fragments of a full length protein or polypeptide to the full length form. For example, the antigen may be less than 5, less than 8, less than 10, less than 15, less than 20, less than 30, less than 50, less than 70, less than 100, or more amino acid residues in length, provided it stimulates a specific immune response.

In certain embodiments the antigen is a cancer antigen. A “cancer antigen” as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen P A et al. (1994) Cancer Res 54:1055-8, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

The terms “cancer antigen” and “tumor antigen” are used interchangeably and refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin and γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancers or tumors and tumor antigens associated with such tumors (but not exclusively), include acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p21 ras), biliary cancer (p21 ras), breast cancer (MUC family; HER2/neu; c-erbB-2), cervical carcinoma (p53; p21 ras), colon carcinoma (p21 ras; HER2/neu; c-erbB-2; MUC family), colorectal cancer (Colorectal associated antigen (CRC)-C017-1A/GA733; APC), choriocarcinoma (CEA), epithelial cell cancer (cyclophilin b), gastric cancer (HER2/neu; c-erbB-2; ga733 glycoprotein), hepatocellular cancer (α-fetoprotein), Hodgkins lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal cancer (HER2/neu; c-erbB-2), squamous cell cancers of cervix and esophagus (viral products such as human papilloma virus proteins), testicular cancer (NY-ESO-1), T-cell leukemia (HTLV-1 epitopes), and melanoma (Melan-A/MART-1; cdc27; MAGE-3; p21ras; gp100^{Pmel117}).

The immunostimulatory ssORN of the invention are generally at least 5 and not more than 100 nucleotides long. In various certain embodiments the immunostimulatory ssORN of the invention are not more than 40 nucleotides long, including specifically 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long.

In certain embodiments the immunostimulatory ssORN of the invention can include a poly-G sequence including at least 4 consecutive G nucleotides, provided that the ssORN is not entirely composed of poly-G. In one embodiment, a poly-G sequence, when present, occupies the 3′ end of the ssORN.

In one embodiment the immunostimulatory ssORN of the invention is not composed entirely of poly-U. In one embodiment the immunostimulatory ssORN of the invention is not composed entirely of poly-A. In one embodiment the immunostimulatory ssORN of the invention is not composed entirely of poly-C.

ssORN of the invention may be of particular use in the treatment of subjects having a cancer, subjects having an infectious disease, subjects having an autoimmune disease, subjects having allergy, and subjects having asthma, but it is not so limited.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

As used herein, a subject having a cancer refers to a subject that has detectable cancerous cells.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

An “infectious disease” as used herein, refers to a disorder arising from the invasion of a host, superficially, locally, or systemically, by an infectious microorganism. Infectious microorganisms include bacteria, viruses, parasites and fungi.

As used herein, a subject having an infectious disease refers to a subject that has been exposed to an infectious organism and has acute or chronic detectable levels of the organism in the body. Exposure to the infectious organism generally occurs with the external surface of the subject, e.g., skin or mucosal membranes and/or refers to the penetration of the external surface of the subject by the infectious organism.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviruses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borrelia burgdorferi, Legionella pneumophilia, Mycobacteria sps (e.g. M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

The ssORN of the invention are also useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

As used herein, an allergy refers to acquired hypersensitivity to a substance (allergen). Allergic conditions include but are not limited to eczema, allergic rhinitis or coryza, hay fever, allergic conjunctivitis, bronchial asthma, urticaria (hives) and food allergies, other atopic conditions including atopic dermatitis; anaphylaxis; drug allergy; and angioedema. Allergic diseases include but are not limited to rhinitis (hay fever), asthma, urticaria, and atopic dermatitis.

As used herein, a subject having an allergy is a subject that has an allergic reaction in response to an allergen.

An allergen refers to a substance (antigen) that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canis (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

As used herein, asthma refers to a disorder of the respiratory system characterized by inflammation, narrowing of the airways, and increased reactivity of the airways to inhaled agents. Asthma is frequently, although not exclusively, associated with an atopic or allergic condition. Symptoms of asthma include recurrent episodes of wheezing, breathlessness, and chest tightness; and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, inosineophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

As used herein, a subject having asthma is a subject that has a disorder of the respiratory system characterized by inflammation, narrowing of the airways and increased reactivity of the airways to inhaled agents. Asthma is frequently, although not exclusively, associated with atopic or allergic symptoms. Asthma is also frequently, although not exclusively, associated with contact with an initiator. An “initiator” as used herein refers to a composition or environmental condition which triggers asthma. Initiators include, but are not limited to, allergens, cold temperatures, exercise, viral infections, SO₂.

ssORN of the invention can be used either alone or combined with other therapeutic agents. The other therapeutic agent in one embodiment is another ssORN of the invention. The ssORN and other therapeutic agent may be administered simultaneously or sequentially. When the other therapeutic agents are administered simultaneously, they can be administered in the same or separate formulations, but are administered at the same time. The other therapeutic agents are administered sequentially with one another and with ssORN, when the administration of the other therapeutic agents and the ssORN is temporally separated. The separation in time between the administration of these compounds may be a matter of minutes or it may be longer. Other therapeutic agents include but are not limited to anti-microbial agents, anti-cancer agents, anti-allergy agents, etc.

The ssORN of the invention may be administered to a subject with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethamine-sulfadoxine, quinacrine HCl, quinine sulfate, quinidine gluconate, spiramycin, stibogluconate sodium (sodium antimony gluconate), suramin, tetracycline, doxycycline, thiabendazole, timidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

The ssORN of the invention may also be administered in conjunction with an anti-cancer therapy. Anti-cancer therapies include cancer medicaments, radiation and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphthalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA, but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vaccine, TA-HPV, TA-CIN, DISC-virus and ImmuCyst/TheraCys, but it is not so limited.

The ssORN of the invention may be administered to a subject with an asthma/allergy medicament. An “asthma/allergy medicament” as used herein is a composition of matter which reduces the symptoms of, prevents the development of, or inhibits an asthmatic or allergic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. In most embodiments the asthma/allergy medicament is useful to some degree for treating both asthma and allergy.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting β₂ agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, bronchodilator/beta-2 agonists, K+ channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors.

Bronchodilator/β₂ agonists are a class of compounds which cause bronchodilation or smooth muscle relaxation. Bronchodilator/β₂ agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, tachyarrhythmias, nausea and vomiting, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β₂ agonists include tachycardia, skeletal muscle tremor, hypokalemia, increased lactic acid, headache, and hyperglycemia.

Conventional methods for treating or preventing allergy have involved the use of anti-histamines or desensitization therapies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, astemizole, azelastine, betatastine, buclizine, ceterizine, cetirizine analogues, CS 560, desloratadine, ebastine, epinastine, fexofenadine, HSR 609, levocabastine, loratidine, mizolastine, norastemizole, terfenadine, and tranilast.

Prostaglandin inducers are compounds which induce prostaglandin activity. Prostaglandins function by regulating smooth muscle relaxation. Prostaglandin inducers include, but are not limited to, S-5751.

The asthma/allergy medicaments also include steroids and immunomodulators. The steroids include, but are not limited to, beclomethasone, fluticasone, triamcinolone, budesonide, corticosteroids and budesonide.

Corticosteroids include, but are not limited to, beclomethasome dipropionate, budesonide, flunisolide, fluticaosone propionate, and triamcinolone acetonide. Although dexamethasone is a corticosteroid having anti-inflammatory action, it is not regularly used for the treatment of asthma/allergy in an inhaled form because it is highly absorbed and it has long-term suppressive side effects at an effective dose. Dexamethasone, however, can be used according to the invention for the treating of asthma/allergy because when administered in combination with nucleic acids of the invention it can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. Barnes & Peterson (1993) Am Rev Respir Dis 148:S1-S26; and Kamada A K et al. (1996) Am J Respir Crit Care Med 153:1739-48.

Systemic corticosteroids include, but are not limited to, methylprednisolone, prednisolone and prednisone. Cortosteroids are associated with reversible abnormalities in glucose metabolism, increased appetite, fluid retention, weight gain, mood alteration, hypertension, peptic ulcer, and aseptic necrosis of bone. These compounds are useful for short-term (3-10 days) prevention of the inflammatory reaction in inadequately controlled persistent asthma. They also function in a long-term prevention of symptoms in severe persistent asthma to suppress and control and actually reverse inflammation. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 974051; July 1997).

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, inosineophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor-mediated mechanisms. Substance P also has been shown to modulate distinct immediate hypersensitivity responses by stimulating the generation of arachidonic acid-derived mediators from mucosal mast cells. McGillies J et al. (1987) Fed Proc 46:196-9 (1987). Substance P is a neuropeptide first identified in 1931. Von Euler and Gaddum J Physiol (London) 72:74-87 (1931). Its amino acid sequence was reported by Chang et al. in 1971. Chang M M et al. (1971) Nature New Biol 232:86-87. The immunoregulatory activity of fragments of substance P has been studied by Siemion I Z et al. (1990) Molec Immunol 27:887-890 (1990).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules do not have a binding strength to the receptor corresponding to the very strong interaction is between the native IgE molecule and its receptor. The molecules having this strength tend to bind irreversibly to the receptor. However, such substances are relatively toxic since they can bind covalently and block other structurally similar molecules in the body. Of interest in this context is that the α chain of the IgE receptor belongs to a larger gene family where, e.g., several of the different IgG Fc receptors are contained. These receptors are absolutely essential for the defense of the body against, e.g., bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

Chromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from inosineophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen. Side effects include drying of the mouth and respiratory secretions, increased wheezing in some individuals, and blurred vision if sprayed in the eyes.

For their use in vitro and in vivo, ssORN of the invention are generally used in an effective amount. As used herein, an effective amount refers generally to any amount that is sufficient to achieve a desired biological effect. In one embodiment an effective amount is a clinically effective amount, wherein a clinically effective amount is any amount that is sufficient to treat a subject having a disease. As used herein, treat and treating refer to reducing, eliminating, or preventing at least one sign or symptom of a disease in a subject having or at risk of having the disease. As used herein, a subject refers to a human or other mammal.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular ssORN being administered, the size of the subject, or the severity of the disease or condition. One of ordinary skill in the art can empirically determine the effective amount of a particular ssORN and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

For any compound described herein the therapeutically effective amount can be initially determined from animal models. A therapeutically effective dose can also be determined from human data for ssORN which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

In order to promote delivery of ssORN into cells, the ssORN optionally can be presented, formulated, or otherwise combined with a cationic lipid. In one embodiment such cationic lipid is DOTAP.

For use in therapy, an effective amount of the ssORN can be administered to a subject by any mode that delivers the ssORN to the desired surface. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

The ssORN of the invention may be delivered to a particular tissue, cell type, or to the immune system, or both, with the aid of a vector. In its broadest sense, a “vector” is any vehicle capable of facilitating the transfer of the compositions to the target cells. The vector generally transports the ssORN, antibody, antigen, and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

As used herein, a “chemical/physical vector” refers to a natural or synthetic molecule, other than those derived from bacteriological or viral sources, capable of delivering the ssORN and/or other medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules. RNA, DNA and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

In one embodiment, the vehicle is a biocompatible microparticle or implant that is suitable for implantation or administration to the mammalian recipient. Exemplary bioerodible implants that are useful in accordance with this method are described in published International Application WO 95/24929, entitled “Polymeric Gene Delivery System”. WO 95/24929 describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the nucleic acid and/or the other therapeutic agent is dispersed throughout a solid polymeric matrix) or a microcapsule (wherein the nucleic acid and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the nucleic acid are administered to the subject via an implant while the other therapeutic agent is administered acutely. Biocompatible microspheres that are suitable for delivery, such as oral or mucosal delivery, are disclosed in Chickering et al. (1996) Biotech Bioeng 52:96-101 and Mathiowitz E et al. (1997) Nature 386:410-414 and PCT Pat. Application WO97/03702.

Both non-biodegradable and biodegradable polymeric matrices can be used to deliver the nucleic acid and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, bacillus Calmette-Guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

For oral administration, the compounds (i.e., ssORN, and optionally other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA, for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the ssORN (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the ssORN (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the ssORN or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the ssORN (or derivatives thereof). The ssORN (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, 111:206-212 (alpha 1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of ssORN (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified ssORN may also be prepared in different formulations depending on the type of chemical modification or the type of device employed.

Formulations suitable for use with a nebulizer, either jet or ultrasonic, will typically comprise ssORN (or derivative) dissolved in water at a concentration of about 0.1 to 25 mg of biologically active ssORN per mL of solution. The formulation may also include a buffer and a simple sugar (e.g., for ssORN stabilization and regulation of osmotic pressure). The nebulizer formulation may also contain a surfactant, to reduce or prevent surface induced aggregation of the ssORN caused by atomization of the solution in forming the aerosol.

Formulations for use with a metered-dose inhaler device will generally comprise a finely divided powder containing the ssORN (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing ssORN (or derivative) and may also include a bulking agent, such as lactose, sorbitol, sucrose, or mannitol in amounts which facilitate dispersal of the powder from the device, e.g., 50 to 90% by weight of the formulation. The ssORN (or derivative) should most advantageously be prepared in particulate form with an average particle size of less than 10 μm (microns), most preferably 0.5 to 5 μm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The ssORN and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of an ssORN and optionally one or more additional therapeutic agents included in a pharmaceutically acceptable carrier.

The therapeutic agent(s), including specifically but not limited to the ssORN, may be provided in particles. Particles as used herein means nano or microparticles (or in some instances larger) which can consist in whole or in part of the ssORN or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the ssORN in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### TLR 7-Independent Recognition of Single-Stranded Phosphodiester RNA

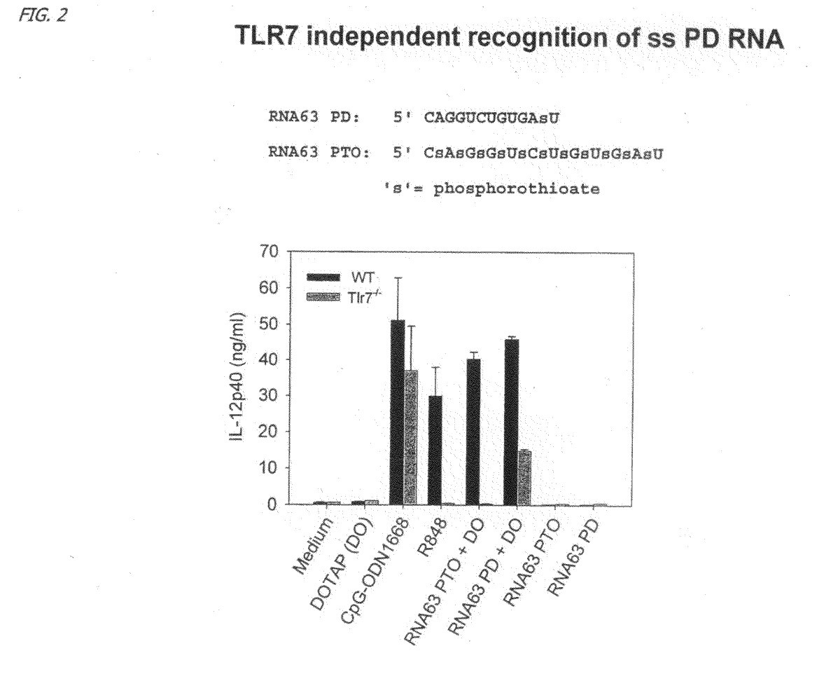

Peripheral blood mononuclear cells were isolated from wild-type and TLR7−/− mice, transferred into suitable growth medium, and then aliquoted separately into individual wells of multiwell culture plates. The following agents were added to individual wells of cells: RNA63 PD, a single-stranded ORN having a nucleotide sequence provided as 5′-CAGGUCUGUGAU-3′ (SEQ ID NO:1), in which all internucleotide linkages are phosphodiester except for a phosphorothioate linkage between the A and U at the 3′ end of the ORN; RNA63 PTO, a single-stranded ORN having the same nucleotide sequence as SEQ ID NO: 1, in which every internucleotide linkage is phosphorothioate; CpG-ODN 1668, an oligodeoxynucleotide having a nucleotide sequence provided as 5′-TCCATGACGTTCCTGATGCT-3′ (SEQ ID NO:2); DOTAP alone; R-848; RNA63 PTO plus DOTAP; RNA63 PD plus DOTAP; and medium alone. Cells were maintained in culture for 24 hours, and then supernatants from individual wells collected and analyzed using enzyme-linked immunosorbent assay (ELISA) specific for IL-12p40. Results are shown in FIG. 2. Data are presented as mean±SEM.

As shown in FIG. 2, both RNA63 PD and RNA63 PTO, when added with DOTAP, induced IL-12p40 in wild-type cells. In contrast, however, RNA63 PD but not RNA63 PTO, when added with DOTAP, induced IL-12p40 in TLR 7−/− cells. This latter result supports the notion that single-stranded ORN with phosphodiester, but not phosphorothioate, backbone induce immune activation in a TLR7-independent manner.

### Example 2

### RNA63 PD Induces IL-12p40 in a Dose-Dependent Manner and Also Induces IL-6 and IFN-α in FLT3-L-Induced Dendritic Cells from TLR 7−/− Mice

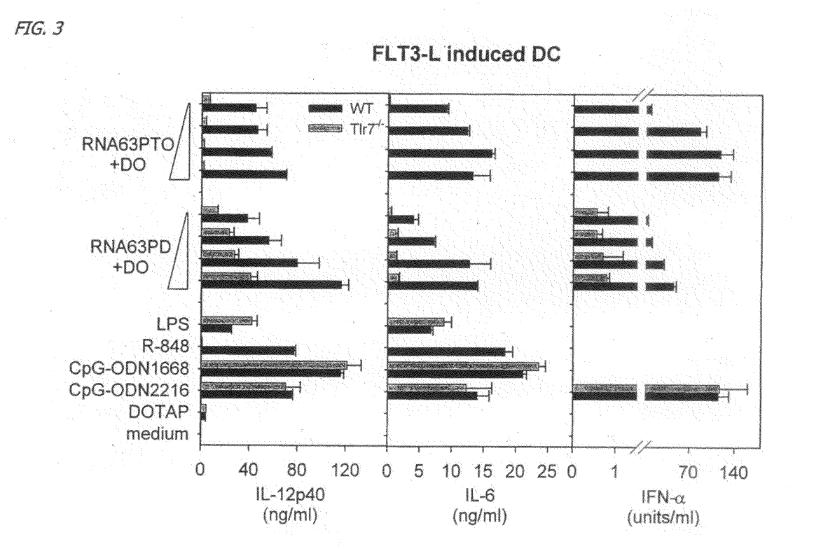

FLT3-L-induced dendritic cells were prepared from wild-type and TLR7−/− mice and cultured in the presence of varied amounts of RNA63 PD or RNA63 PTO, each with DOTAP. After 24 hours incubation, supernatants were collected and analyzed by ELISA for IL-12p40, IL-6, and IFN-α. LPS, R-848, CpG-ODN 1668, CpG-ODN 2216 (5′-GGGGGACGATCGTCGGGGG-3′, SEQ ID NO:3), DOTAP alone, and medium alone were run as controls. Results are shown in FIG. 3. Data are presented as mean±SEM.

As shown in the figure, both RNA63 PD and RNA63 PTO, each in the presence of DOTAP, induced significant amounts of IL-12p40 (FIG. 3A), IL-6 (FIG. 3B), and IFN-α (FIG. 3C) in wild-type dendritic cells. In contrast, however, RNA63 PD but not RNA63 PTO, when added with DOTAP, induced IL-112p40 and, albeit less strongly, both IL-6 and IFN-α in TLR7−/− dendritic cells. The amount of IL-12p40 induced by RNA63 PD in TLR7−/− dendritic cells varied in proportion to the concentration of RNA63 PD.

### Example 3

### RNA63 PD, But Not RNA63 PTO, Induces CD69 on FLT3-L-Induced Dendritic Cells from TLR7−/− Mice

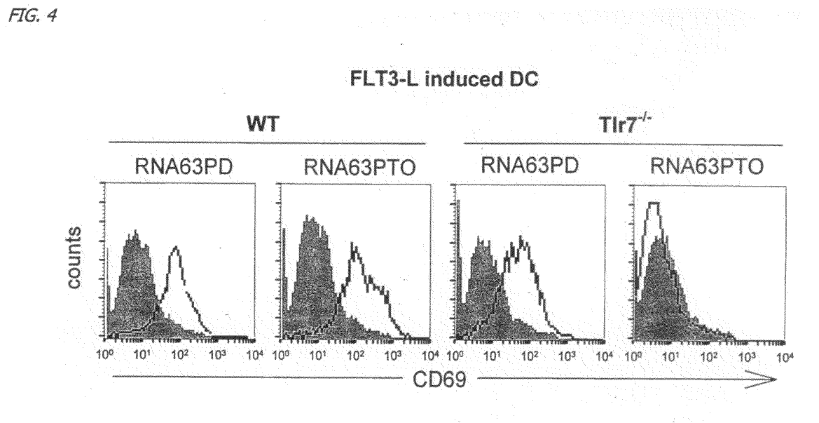

FLT3-L-induced dendritic cells were prepared from wild-type and TLR7−/− mice and cultured for 24 hours in the presence of RNA63 PD or RNA63 PTO, each with DOTAP, as in Example 2. After 24 hours incubation, cells were collected and analyzed by FACS for CD69. Results are shown in FIG. 4.

As shown in FIG. 4, both RNA63 PD and RNA63 PTO, each in the presence of DOTAP, induced significant amounts of CD69 in wild-type dendritic cells (left two panels). In contrast, however, RNA63 PD but not RNA63 PTO, when added with DOTAP, induced CD69 on TLR7−/− dendritic cells (right two panels).

### Example 4

### RNA63 PD, But not RNA63 PTO, Induces IL-12p40 in M-CSF-Derived Macrophages and GM-CSF-Derived Dendritic Cells from TLR7−/− Mice

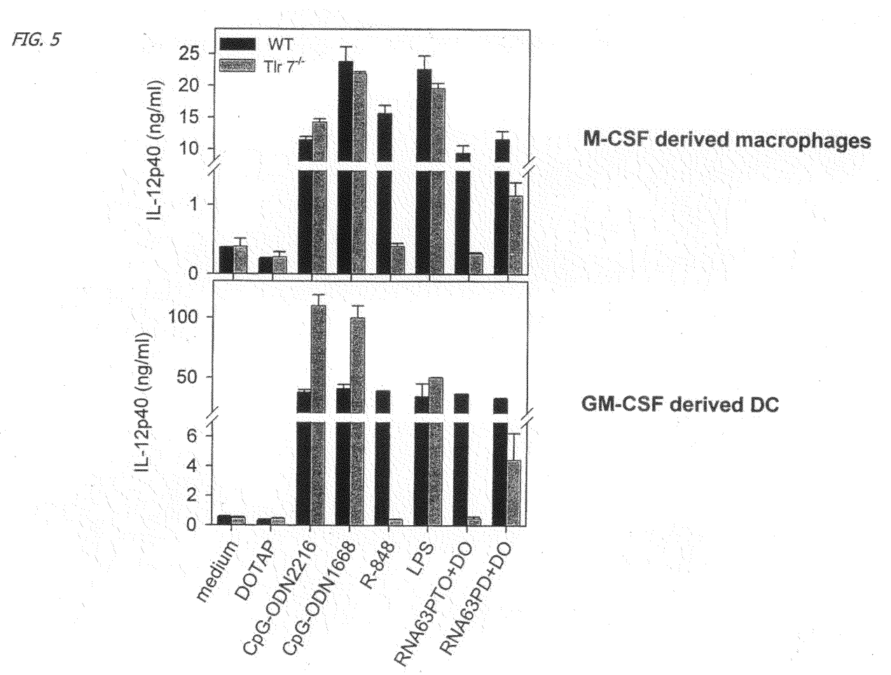

M-CSF-derived macrophages and GM-CSF-derived dendritic cells were prepared from wild-type and TLR7−/− mice and cultured for 24 hours in the presence of RNA63 PD plus DOTAP, RNA63 PTO plus DOTAP. LPS, R-848, CpG-ODN 1668, CpG-ODN 2216, DOTAP alone, or medium alone. Culture supernatants were then collected and analyzed using ELISA for IL-12p40. Results are shown in FIG. 5. Data are presented as mean±SEM.

As shown in FIG. 5, both RNA63 PD and RNA63 PTO, each in the presence of DOTAP, induced significant amounts of IL-12p40 in both M-CSF-derived macrophages and GM-CSF-derived dendritic cells from wild-type mice. In contrast, however, RNA63 PD but not RNA63 PTO, when added with DOTAP, induced significant amounts of IL-12p40 in TLR7−/− M-CSF-derived macrophages and TLR7−/− GM-CSF-derived dendritic cells.

### Example 5

### TLR7-Independent Recognition of Single-Stranded Phosphodiester RNA is MyD88-Dependent

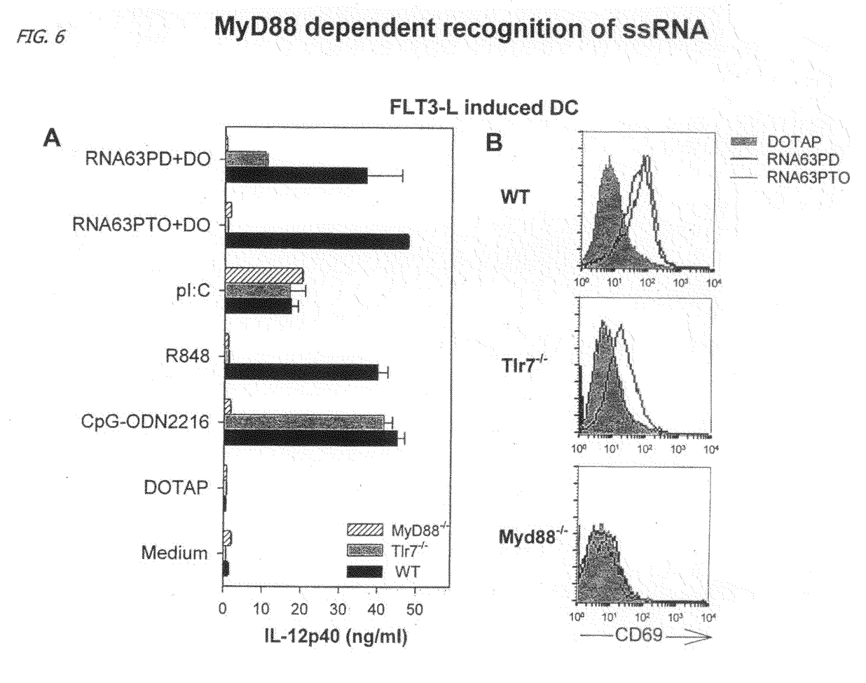

FLT3-L-induced dendritic cells were separately prepared from wild-type, TLR7−/−, and MyD88−/− mice and cultured for 24 hours in the presence of RNA63 PD or RNA63 PTO, each with DOTAP, as in Example 2. After 24 hours incubation, cells were collected and analyzed by ELISA for IL-12p40 and by FACS for CD69. Results are shown in FIG. 6.

As shown in FIG. 6, induction of IL-12p40 and CD69 was absent in MyD88−/− dendritic cells for RNA63 PD and for RNA63 PTO alike.

### Example 6

### ssRNA-Driven TLR 7-Independent Immune Stimulation is Dependent on TLR9 and TLR8

Since the ssRNA-driven TLR7-independent immune stimulation is dependent on MyD88 and endosomal maturation, it was hypothesized that other intracellular TLRs are involved. TLR3 seems a poor candidate for the recognition of ssRNA since it mainly relies on the adaptor molecule TRIF and not MyD88 (Hoebe, Du et al. 2003). Nevertheless, to rule out TLR3 as receptor for ssRNA, TLR3- and TLR7-deficient mice were crossed to obtain TLR3/TLR7 double deficient mice, and immune cells from these mice were tested for ssRNA stimulation. Results of these experiments showed that RNA63-driven IL-12p40 production and CD69 upregulation in TLR3/TLR7 double deficient FLT3-L-induced DCs were still functional, suggesting a lack of involvement of TLR3 in the recognition of ssRNA in a TLR7-independent manner. TLR7/TLR9 double deficient mice were also generated to investigate the involvement of TLR9 in RNA63 recognition. Interestingly, TLR7/TLR9 double deficient GM-CSF-derived DCs and sorted mDCs failed to produce IL-12p40 and to upregulate CD69, suggesting the involvement of TLR9 in the recognition of ssRNA in a TLR7-independent manner. Overall, these data suggest that TLR9 is cross-reactive to ssRNA and mediates immune stimulation of ssRNA.

### Example 7

### Role of TLR9 in the Recognition of Phosphodiester ssRNA

To further investigate the role of TLR9 in the recognition of PD ssRNA, various RNA sequences, such as RNA41 (5′-GCCCGACAGAAGAGAGACAC-3′; SEQ ID NO:4) and RNA42 (5′-ACCCAUCUAUUAUAUAACUC-3′; SEQ ID NO:5) that have been previously described as not active in murine cells when synthesized with a PTO backbone (Heil, Hemmi et al. 2004), were synthesized. Comparing the stimulatory capacity of ORN with a PTO or PD backbone it was observed that RNA41 PD and RNA42 PD stimulate IL-12p40, IL-6, and IFN-α in a TLR7-independent fashion in murine cells and TNF-α (but no IFN-α) in human PBMCs, whereas the corresponding PTO-modified ORN were inactive. Interestingly, when wild-type, TLR7-deficient, or TLR9-deficient mDC were stimulated with RNA41 PD and RNA42 PD, TLR9-deficient cells failed to produce IL-12p40, suggesting that TLR9 and not TLR7 is involved in the recognition of non-G/U-rich ssRNA molecules. In contrast, GU-rich RNA40 (5′-GCCCGUCUGUUGUGUGACUC-3′; SEQ ID NO:6) induced some IL-12p40 in TLR7-single deficient and in TLR9-single deficient cells, suggesting that TLR7 or TLR9 can function as ssRNA receptor. pDC recognized the GU-rich RNA in a TLR7-dependent fashion, but surprisingly the non-GU-rich ORN RNA41 and RNA42 were also recognized by wild-type cells, although TLR7-single deficient or TLR9-single deficient cells failed to respond, suggesting that TLR7/TLR9 complexes are involved in the recognition of non-GU-rich RNAs.

### Example 8

### Role of TLR8 in the Recognition of Phosphodiester ssRNA

Using mTLR8-specific siRNA it was demonstrated that TLR8 is involved in the recognition of ssRNA. Wild-type FLT3-L-induced DCs were treated with TLR8-specific siRNA or a control siRNA against eGFP and stimulated with CpG-ODN, RNA40 PD, RNA41 PD, and RNA42 PD. mTLR8-specific siRNA-treated cells showed a 75% or 50% reduction in IL-12 secretion upon RNA41 and RNA42 stimulation, respectively. RNA40 stimulation was not influenced by mTLR8-specific siRNA since RNA40-driven immune stimulation is critically dependent on mTLR7. The control eGFP-specific siRNA had no inhibitory effect on IL-12 production and IL-112 values corresponded with stimulation of non-siRNA-transfected cells. To further assess the involvement of TLR8 in the TLR7-independent ssRNA-driven immune stimulation, TLR7-deficient FLT3-L-induced DCs were transfected with mTLR8- or eGFP-specific siRNA and the RNA63-induced IL-12p40 production determined. DCs treated with mTLR8-specific siRNA showed a strong reduction in IL-12p40 production and downregulation of activation markers such as CD40, whereas eGFP-specific siRNA had no effect. The reduction in RNA63-driven immune activation correlated with siRNA-driven downregulation of mTLR8 RNA. Taken together, these data demonstrate that TLR8 is involved in the recognition of ssRNA.

### Example 9

### TLR8/TLR9 Heterodimers Mediate ssRNA Recognition

Since TLR8 and TLR9 are involved in the recognition of ssRNA, it was investigated if both receptors cooperate and would form complexes to mediate ssRNA recognition. By transfecting tagged TLRs into HEK293 cells with subsequent immunoprecipitation and western blotting, TLR8-flag was shown to associate with mTLR9-HA, and vice versa, whereas the intracellular TLR3 and TLR9 did not interact.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of inducing a Th1-like immune response in a subject, the method comprising
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

2. A method of inducing an antigen-specific response in a subject, the method comprising
administering to the subject an antigen; and
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

3. A method of treating a subject having a cancer, the method comprising
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

4. A method of treating a subject having an infectious disease, the method comprising
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

5. A method of treating a subject having an allergic condition, the method comprising
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

6. A method of treating a subject having asthma, the method comprising
administering to the subject an effective amount of an immunostimulatory single-stranded oligoribonucleotide (ssORN) 5-100 nucleotides long, wherein the immunostimulatory ssORN has a phosphodiester backbone and comprises a nucleotide sequence that
(1) is free of guanosine (G), or
(2) comprises at least one G, with proviso that when the nucleotide sequence comprises at least one G, the nucleotide sequence is
(a) free of uridine (U), and
(b) free of CpG motif X₁X₂CGX₃X₄, wherein X₁, X₂, X₃, and X₄ are nucleotides and CG is a cytosine (C)-guanine (G) dinucleotide, wherein the C of the CG dinucleotide is unmethylated,
with proviso that the immunostimulatory ssORN is not present as part of a double-stranded ribonucleic acid (RNA) molecule.

7. The method of any one of claims 1-6, wherein the immunostimulatory ssORN is 5-40 nucleotides long.

8. The method of any one of claims 1-6, wherein the immunostimulatory ssORN is 5-20 nucleotides long.

9. The method of any one of claims 1-6, wherein the immunostimulatory ssORN is 5-12 nucleotides long.

10. The method of any one of claims 1-6, wherein the immunostimulatory ssORN is a synthetic ssORN.

11. The method of any one of claims 1-6, wherein the immunostimulatory ssORN is not a poly-nucleotide selected from the group consisting of poly-U, poly-G, poly-A, or poly-C.

12. The method of any one of claims 1-6, wherein the administering to the subject the effective amount of the immunostimulatory ssORN is systemically administering to the subject an effective amount of the immunostimulatory ssORN.
